(19) United States
(12) Patent Application Publication
(10) Pub. No.: US 2015/0182117 A1
(43) Pub. Date: Jul. 2, 2015

(54) METHOD FOR MAINTAINING GEOMETRIC ALIGNMENT OF SCANS IN CASES OF STRONG PATIENT MOTION

(71) Applicant: KONINKLIJKE PHILIPS N.V., EINDHOVEN (NL)

(72) Inventors: Julien Senegas, Hamburg (DE); Peter Koken, Hamburg (DE); Torbjørn Vik, Hamburg (DE)

(21) Appl. No.: 14/412,369

(22) PCT Filed: Jun. 28, 2013
(86) PCT No.: PCT/IB2013/055324

§ 371 Date: Dec. 31, 2014

Related U.S. Application Data
(60) Provisional application No. 61/668,155, filed on Jul. 5, 2012.

(51) Int. Cl. A61B 5/00; A61B 5/055

(52) U.S. Cl. CPC A61B 5/0037; A61B 5/055; A61B 5/7246; A61B 5/742; A61B 2576/00

(57) ABSTRACT

A magnetic resonance (MR) system (10) and method (100) maintains geometric alignment of diagnostic scans during an examination of a patient (12). At least one processor (40) is programmed to, in response to repositioning of the patient (12) during the examination, perform an updated survey scan of the patient (12). A scan completed during the examination is selected as a template scan. A transformation map between the template scan and the updated survey scan is determined using a registration algorithm, and the transformation map is applied to a scan geometry of a remaining diagnostic scan of the examination. A scan plan for the remaining diagnostic scan is generated using the updated scan geometry. The remaining diagnostic scan is performed according to the scan plan.

The present application relates generally to magnetic resonance (MR) imaging. It finds particular application in conjunction with maintaining geometric alignment of scans in cases of strong patient motion and will be described with particular reference thereto. However, it is to be understood that it also finds application in other usage scenarios and is not necessarily limited to the aforementioned application.

An MR examination generally includes of a list of scans with different MR contrasts and/or different orientations. For the reading radiologist, it is often desirable to compare these different scans slice by slice, for example, to analyze the appearance of a lesion under different MR contrast types. If the position of the patient changes between two consecutive scans, the same voxel in the scans before and after motion will point to different locations within the body of the patient. This hampers slice-by-slice comparison of these scans and makes diagnosis more difficult and less reliable. Further, post-processing applications, such as subtraction of pre-contrast and post-contrast scans, may also be prevented from being performed.

Deviations between scans are particularly severe when the patient is repositioned by the operator during examination. Deviations between scans may also be severe when the patient feels uncomfortable in the scanner bore, the technical assistant has forgotten to put a sensor (e.g., a breathing bell) that is required for a particular scan, or there is a technical problem with, for example, a coil.

When an examination has been interrupted due to repositioning of the patient, it is currently difficult to resume the examination while maintaining the geometric alignment of the scans before and after repositioning. The technical assistant may decide, in some cases, to repeat the whole examination, which leads to time delays and increased time pressure. Alternatively, after the completion of the examination, it is also possible to register the reconstructed images and realign the images acquired before and after repositioning. However, in this case, the image quality of the registered scans is lower due to increased partial volume effects.

In examinations that use automated scan planning software, it is possible to mitigate the effects of repositioning by re-launching the automated scan planning functionality. Generally, this will require the acquisition of a new survey scan as a basis for the planning Re-launching the automated scan planning functionality advantageously ensures alignment between the initial and the repeated survey scan. However, it does not necessarily ensure alignment between the diagnostic scans acquired before and after repositioning.

The present application provides a new and improved system and method which overcome the above-referenced problems and others.

In accordance with one aspect, a magnetic resonance (MR) system for maintaining geometric alignment of diagnostic scans during an examination of a patient is provided. The system includes at least one processor programmed to, in response to repositioning of the patient during the examination, perform an updated survey scan of the patient. A scan completed during the examination is selected as a template scan, and a transformation map between the template scan and the updated survey scan is determined using a registration algorithm. The transformation map is applied to an initially defined scan geometry of a remaining diagnostic scan of the examination. A scan plan for the remaining diagnostic scan is generated using the updated scan geometry. The remaining diagnostic scan is performed according to the scan plan.

In accordance with one aspect, a magnetic resonance (MR) method for maintaining geometric alignment of diagnostic scans during an examination of a patient is provided. The method is performed in response to repositioning of the patient during the examination. It includes performing by at least one processor an updated survey scan of the patient. A scan completed during the examination is selected as a template scan, and a transformation map between the template scan and the updated survey scan is determined using a registration algorithm. The transformation map is applied to a initially defined scan geometry of a remaining diagnostic scan of the examination. A scan plan for the remaining diagnostic scan is generated using the updated scan geometry. The remaining diagnostic scan is performed according to the scan plan.

In accordance with one aspect, a magnetic resonance (MR) system for maintaining geometric alignment of diagnostic scans of a patient is provided. The system includes an MR scanner controlled to perform scans of the patient and at least one processor programmed to perform an updated survey scan of the patient using the MR scanner. A completed scan of the patient is selected as a template scan. A transformation map between the template scan and the updated survey scan is determined. A scan plan for the diagnostic scan is generated using the transformation map. The diagnostic scan is performed according to the scan plan.

One advantage resides in geometric alignment of scans before and after patient repositioning.

Another advantage resides in geometrically aligned scans acquired before and after patient repositioning that do no exhibit partial volume effects.

Another advantage resides in geometrically aligned scans before and after patient repositioning without loss of image quality.

Another advantage resides in geometrically aligned scans notwithstanding strong patient motion.

Still further advantages of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

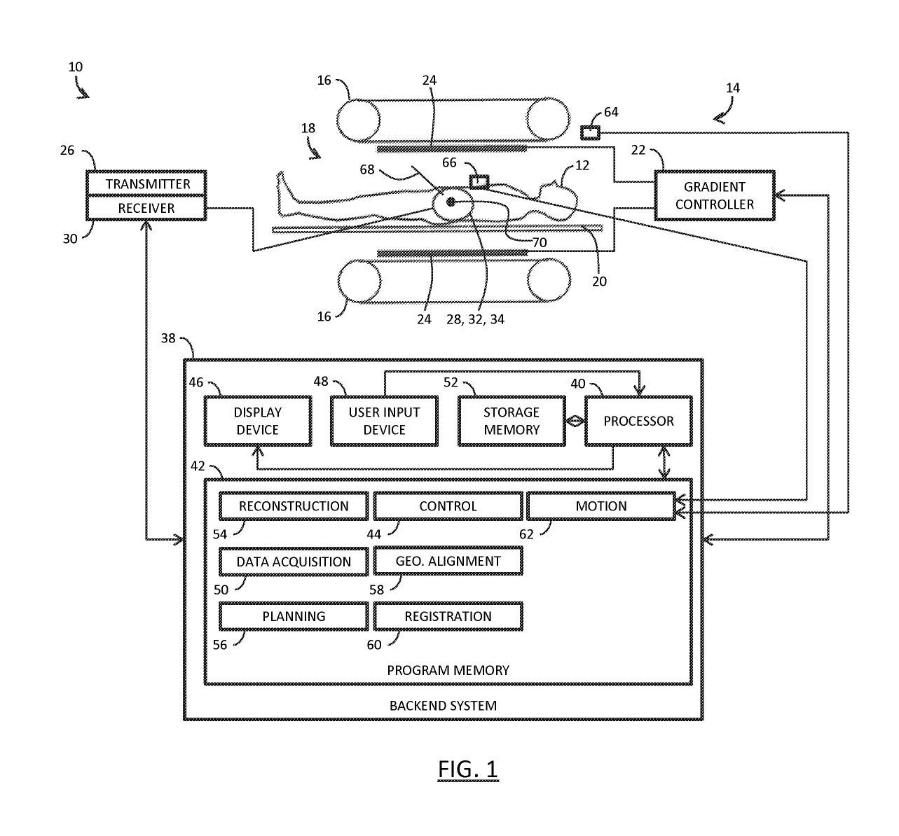

FIG. 1 illustrates a magnetic resonance (MR) system for geometric alignment between scans.

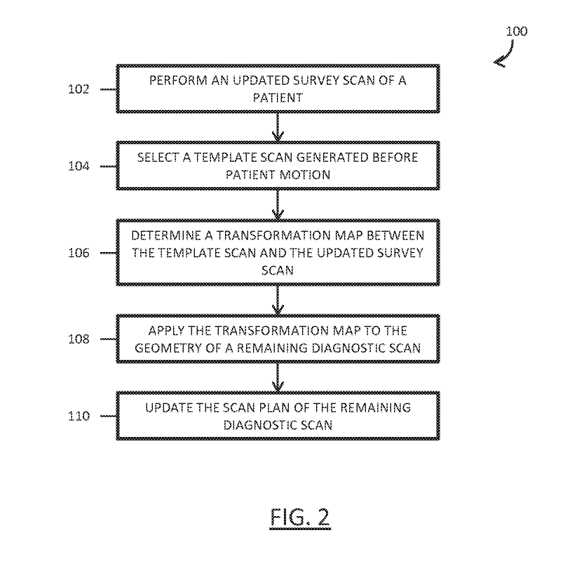

FIG. 2 illustrates a method for geometrically aligning a diagnostic scan performed after patient motion to a diagnostic scan performed before the patient motion.

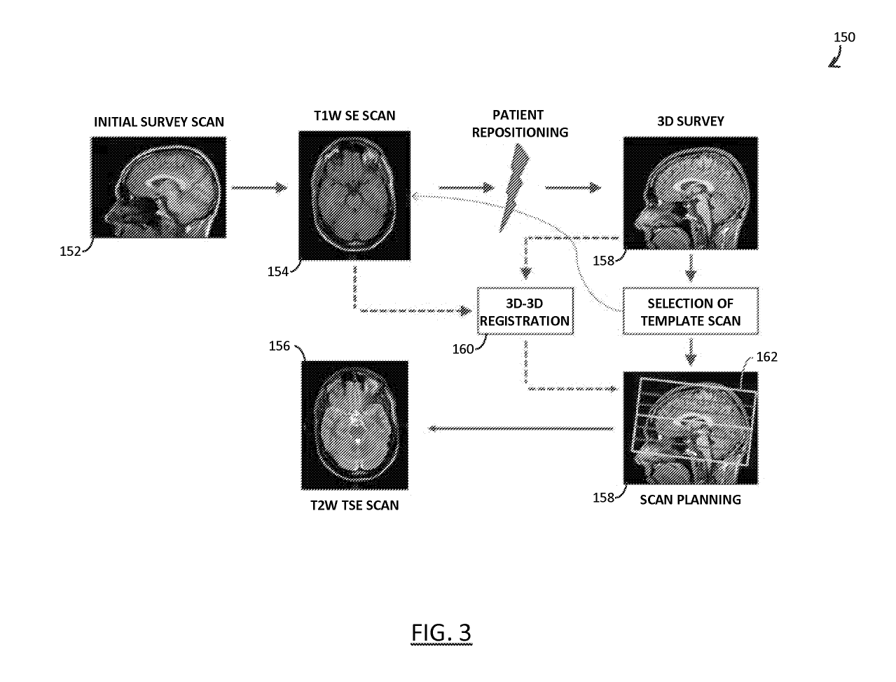

FIG. 3 illustrates an example of an examination with patient motion.

With reference to FIG. 1, a magnetic resonance (MR) system 10 utilizes MR to perform an examination of a patient 12. The MR system 10 includes a scanner 14. The scanner 14 includes a main magnet 16 that creates a strong, static B₀ magnetic field extending through an examination volume 18. The examination volume 18 is sized to accommodate the patient 12, which is positioned in the examination volume 18 during the examination. A patient support 20 supports the patient 12 in the scanner 14 and facilitates positioning the patient 12 in the examination volume 18.

The main magnet 16 typically employs superconducting coils to create the static B₀ magnetic field. However, the main magnet 16 can also employ permanent or resistive magnets. Insofar as superconducting coils are employed, the main magnet 16 includes a cooling system, such as a liquid helium cooled cryostat, for the superconducting coils. The strength of the static Bₒ magnetic field is commonly one of 0.23 Tesla, 0.5 Tesla, 1.5 Tesla, 3 Tesla, 7 Tesla, and so on in the examination volume 18, but other strengths are contemplated.

As illustrated, the main magnet 16 is an open type and includes two superconducting coils spaced apart to define the examination volume 18. The superconducting coils produce the static Bₒ magnetic field similar to the way in which a Helmholtz coil would. The advantage of an open magnet is that it provides easy access to the patient 12. However, different types of main magnets can also be employed. For example, a split cylindrical main magnet and/or a cylindrical main magnet can be employed. A split cylindrical main magnet is similar to a cylindrical main magnet, which includes a cryostat, except that the cryostat is split into two sections to allow access to the iso-plane of the magnet.

A gradient controller 22 of the scanner 14 is controlled to superimpose magnetic field gradients, such as x, y and z gradients, on the static B₀ magnetic field in the examination volume 18 using a plurality of magnetic field gradient coils 24 of the scanner. The magnetic field gradients spatially encode magnetic spins within the examination volume 18. Typically, the plurality of magnetic field gradient coils 24 include three separate magnetic field gradient coils spatially encoding in three orthogonal spatial directions.

Further, one or more transmitters 26, such as a transceiver, of the scanner 14 are controlled to transmit B₁ resonance excitation and manipulation radio frequency (RF) pulses into the examination volume 18 with one or more transmit coils 28, such as a whole body coil and/or a surface coil, of the scanner 14. The B₁ pulses are typically of short duration and, when taken together with the magnetic field gradients, achieve a selected manipulation of magnetic resonance. For example, the B₁ pulses excite the hydrogen dipoles to resonance and the magnetic field gradients encode spatial information in the frequency and phase of the resonance signal. By adjusting the RF frequencies, resonance can be excited in other dipoles, such as phosphorous, which tend to concentrate in known tissues, such as bones.

One or more receivers 30, such as a transceiver, of the scanner 14 are controlled to receive spatially encoded magnetic resonance signals from the examination volume 18 and demodulate the received spatially encoded magnetic resonance signals to MR data sets. The MR data sets include, for example, k-space data trajectories. To receive the spatially encoded magnetic resonance signals, the receivers 30 use one or more receive coils 32, such as a whole body coil and/or a surface coil, of the scanner 14. The receivers 30 typically store the MR data sets in a buffer memory.

As illustrated, the transmit coils 28 and the receive coils 32 include a surface coil 34 positioned on the surface of the subject 12. The surface coil 34 is employed as both a transmit coil and a receive coil. However, it is to be appreciated that the surface coil 34 can be employed as only one of a transmit coil and a receive coil.

A backend system 38 coordinates the examination of the patient 12. The examination includes performing a plurality of diagnostic scans of the patient 12, typically with different contrasts and/or different orientations. The backend system 38 includes at least one processor 40 and at least one program memory 42. The program memory 42 includes processor executable instructions that, when executed by the processor 40, coordinate the examination of the patient 12. The processor 40 executes the processor executable instructions to coordinate the examination of the patient 12.

A control module 44 of the processor executable instructions controls overall operation of the backend system 38. The control module 44 suitably displays a graphical user interface (GUI) to an operator of the backend system 38 using a display device 46 of the backend system 38. Further, the control module 44 suitably allows the operator to interact with the GUI using a user input device 48 of the backend system 38. For example, the operator can interact with the GUI to instruct the backend system 38 to coordinate the examination of the patient 12.

A data acquisition module 50 of the processor executable instructions performs the diagnostic scans of the patient 12. For each diagnostic scan, the data acquisition module 50 receives a scan plan. The scan plan includes one or more scan parameters, such as number of slices, and one or more imaging sequences. An imaging sequence defines a sequence of B₁ pulses and/or magnetic field gradients that produce spatially encoded MR signals from the examination volume 18. The scan plan defines the geometry of the diagnostic scan, such as slice positioning. The data acquisition module 50 then controls the transmitters 26 and/or the gradient controller 22 according to the scan parameters to implement the imaging sequence within the examination volume 18. Further, the data acquisition module 50 controls the receivers 30 according to the scan parameters to capture the spatially encoded MR signals to an MR data set. The MR data set is typically stored in at least one storage memory 52 of the backend system 38.

A reconstruction module 54 of the processor executable instructions reconstructs the MR data sets of the diagnostic scans into MR images or maps of the subject 12. This includes, for each MR signal captured by the MR data sets, spatially decoding the spatial encoding by the magnetic field gradients to ascertain a property of the MR signal from each spatial region, such as a pixel or voxel. The intensity or magnitude of the MR signal is commonly ascertained, but other properties related to phase, relaxation time, magnetization transfer, and the like can also be ascertained. The MR images or maps are typically stored in the storage memory 52.

A planning module 56 of the processor executable instructions plans the diagnostic scans. This includes performing an initial survey scan, such as a fast, three-dimensional (3D) survey scan, of the patient 12 using the data acquisition module 50. Based on the initial survey scan, the planning module 56 determines the scan plan, including the scan parameters and the imaging sequences, for each of the diagnostic scans. Determining the scan plan for the diagnostic scan includes determining the geometry of the diagnostic scan. The geometry of the diagnostic scan can be determined automatically and/or manually with reference to the coordinate frame of the initial survey scan.

To manually determine the geometry of the diagnostic scan, the initial survey scan is displayed on the GUI with a scan geometry overlaid thereon. The scan geometry can be a default scan geometry or determined automatically, as described below. Further, the scan geometry can, for example, be represented by the positions of slices. An operator of the system then manipulates the scan geometry using the user input device 48 as desired. Manipulation includes, for example, positioning and/or sizing.

To automatically determine the geometry of the diagnostic scan, the planning module 56 uses an examination template specifying the geometries of the diagnostic scans in relation to patient landmarks. The planning module 56 analyzes the initial survey scan, within image-space or k-space, to locate the patient landmarks and then, based on the examination template, determines the geometries of the diagnostic scans relative to the landmarks. The examination template is, for example, stored in the storage memory 52 and generated prior to beginning the examination.

A geometric alignment module 58 of the processor executable instructions, in response to patient motion between the diagnostic scans, updates the scan plans of the one or more remaining diagnostic scans to ensure alignment of the geometries of the remaining diagnostic scans with the geometries of the completed diagnostic scans. This is important because, if the position of the patient 12 changes between two consecutive diagnostic scans, the same voxel in the two diagnostic scans will point to different locations within the patient 12. Suitably, the scan plans of the remaining diagnostic scans are updated right before performing the corresponding diagnostic scans in case there is additional patient motion.

With reference to FIG. 2, the processor 40 is controlled to perform a method 100 for updating the scan plan of a diagnostic scan after patient motion is detected. The geometric alignment module 58 suitably performs the method 100 for each of the remaining diagnostic scans to update the scan plans of the remaining diagnostic scans. The method 100 includes performing 102 an updated survey scan, such as a fast, three-dimensional (3D) survey scan, of the patient 12 using the data acquisition module 50. The updated survey scan is generally short, such as less than a minute. Further, the contrast type of the updated survey scan can, but need not, be selected to match the contrast type of the initial survey scan.

A template scan is further selected 104 from the one or more diagnostic scans, or the one or more survey scans, completed before the patient motion. Further, the contrast type of the template scan can, but need not, match the contrast type of the updated survey scan. For example, the survey scans and the template scan can have different contrast types. Further, the selection can be performed manually and/or automatically. As to manual selection, an operator of the system 10 selects one of the completed diagnostic scans, or one of the completed survey scans, to employ as the template scan using, for example, the user input device 48. The completed diagnostic scans or the completed survey scans can be displayed on the GUI to facilitate selection. As to automatic selection, any number of selection criteria can be employed. For example, the most recently completed diagnostic scan can be employed. As another example, the initial survey scan can be employed. This may be advantageous for registration, discussed hereafter, because the updated survey scan can be chosen to have a similar MR contrast as the initial survey scan. However, this may not lead to optimal alignment in cases where there was some patient motion between the initial survey scan and the completed diagnostic scans.

A registration module 60 of the processor executable instructions is employed to determine 106 a transformation map between the template scan and the updated survey scan within image-space or k-space. Any registration algorithm can be employed. However, a rigid registration algorithm is preferably employed, since no severe anatomical changes occur before and after repositioning and only rigid transformations, such as rotations and translations, can be taken into account for scan planning Further, rigid registration algorithms are generally faster and more robust than affine or elastic registration algorithms.

Since the quality of the registration depends on the similarity of the template scan and the updated survey scan, and of the quality metrics used in the registration algorithm, the registration algorithm can be adapted to the MR contrast types of the template scan and of the updated survey scan. For example, if the template scan and the updated survey scan have similar MR contrasts, a cross-correlation based registration algorithm can be applied. However, if the template scan and the updated survey scan have different contrast types, a mutual-information based registration algorithm can be applied.

The transformation map is applied 108 to the geometry of the diagnostic scan, and the planning module 56 is employed to update 110 the initially defined scan plan for the diagnostic scan using the updated survey scan and the updated geometry. The updating of the geometry can be automatic or subject to operator supervision. For example, the planning module 56 displays the updated survey scan on the GUI with the updated scan geometry overlaid thereon. An operator of the system 10 can then manipulate the scan geometry using the user input device 48 as desired. Once the desired scan geometry is achieved, the planning module 56 updates the scan plan of the diagnostic scan according to the manipulated scan geometry.

It is to be appreciated that when the remaining diagnostic scans include a plurality of diagnostic scans, the method 100 is performed multiple times, once for each of the remaining diagnostic scans. While each step of the method 100 can be performed for each of the remaining diagnostic scans, in some embodiments, steps are only performed as needed. For example, the selection of the template scan can only be performed once for all of the remaining diagnostic scans. As another example, the updated survey scan can only be performed if there was additional patient motion from the time of generating the last survey scan to the present time. As another example, the transformation map is only determined if a transformation map has not been previously generated with the combination of the selected template scan and the updated survey scan.

Referring back to FIG. 1, the geometric alignment module 58 can further verify geometric consistency between a reference scan acquired before repositioning and an updated survey scan. The reference scan typically includes one or more low-resolution MR anatomical images with a given MR contrast type, which can be processed to compute a map of a desired quantity, such as receive sensitivity, transmit sensitivity, B0, and so on. The reference scan can, for example, be a receive coil sensitivity scan, as performed during an examinations based on a multi-channel receive coil, or a transmit coil sensitivity scan, as performed during an examination using a multi-channel transmit coil. The reference scan can also, for example, be a B0 or B1 mapping scan.

To verify geometric consistency, Actions 102 to 106 of FIG. 2 are performed, as described above. The template scan can, for example, be the reference scan. The transformation map is then analyzed to determine the extent of the transformation. Insofar as the extent of the transformation is less than a threshold, the reference scan is registered to the updated survey scan, as described. Otherwise, an operator of the system 10 can be asked to repeat the reference scan using, for example, the GUI or the reference scan can be automatically repeated. To repeat the reference scan, the scan plan of the reference scan is updated to using the updated survey scan and the reference scan is repeated with the updated scan plan.

Alternatively, an updated survey scan is performed, as described above. The reference scan is then registered to the updated survey scan, as described below. The registered reference scan is compared with the updated survey scan. Insofar as the reference scan is not geometrically consistent, an operator of the system 10 can be asked to repeat the reference scan using, for example, the GUI or the reference scan can be automatically repeated.

The geometric alignment 58 module can further register the reference scan to the updated survey scan so that scans acquired after repositioning can use the reference scan.

Registering the reference scan to the updated survey scan includes determining a transformation map between the reference scan and the updated survey scan, as described above in FIG. 2. In determining the transformation map, raw image data (i.e., directly obtained from the k-space data by means of Fourier transform) acquired during the reference scan, or a map computed from the raw image data, is employed. It is, however, preferred to use the raw image data, since the computed map often does not contain anatomical information anymore, and is thus not well suited for registration with the updated survey scan.

A motion module 62 of the processor executable instructions detects patient motion. Motion can be detected manually and/or automatically. As to manual detection, an operator of the system 10 specifies when there is patient motion, for example, using the user input device 48. As to automatic detection, any number of well-known approaches for detecting motion can be employed. For example, before each full MR scan, an image or map of the patient is generated using a camera 64, a fast MR scan, or the like. A current image or map is then compared with a reference image or map generated at the beginning of the examination to detect motion. As another example, before each full MR scan, the locations of electromagnetic transponders 66 positioned on the patient are determined. Current locations are then compared with reference locations determined at the beginning of the examination to detect motion. Navigator sequences can also be used to detect patient motion.

With reference to FIG. 3, an example 150 of an examination with patient repositioning is provided. The examination begins with an initial survey scan 152 of the patient 12. Thereafter, a first diagnostic scan 154 is planned using the initial survey scan 152 and then performed. The first diagnostic scan 154 is, for example, a Ti weighted spin echo scan. Sometime between completing the first diagnostic scan 154 and beginning a second diagnostic scan 156, the patient is repositioned.

To ensure geometric alignment between the first diagnostic scan 154 and the second diagnostic scan 156, the method 100 of FIG. 2 is performed. As discussed above, this includes performing an updated survey scan 158, such as a 3D scan. As can be seen by comparing the initial survey scan 152 and the updated survey scan 158, the position of the patient 12 has slightly changed (i.e., a translation in the head direction). The first diagnostic scan 154 is then selected as a template scan, and a transformation map between the updated survey scan 158 and the template scan 154 is determined 160 using a registration algorithm, such as a 3D rigid registration algorithm. The transformation map is applied to the scan geometry of the second diagnostic scan 156, and the updated scan geometry 162 is overlaid on a visualization of the updated survey scan 158. The updated scan geometry 162 is represented by the updated positions of the slices of the second diagnostic scan 156. An operator of the system 10 approves the updated scan geometry 162, and the scan plan of the second diagnostic image 156 is updated with the updated scan geometry 162. The second diagnostic scan 156 is then performed using the updated scan plan. The second diagnostic scan 156 is, for example, a T2 weighted turbo spin echo scan.

The above described approach to maintaining geometric alignment finds application in any MR clinical applications, including MR-guided interventions and MR-guided radiation therapy. For example, as shown in FIG. 1, MR is employed to guide a shaft or needle 68 to a target 70 within the patient 12. Namely, MR images of the shaft or needle 68 can be displayed on, for example, the display device 46, to the interventionist while they insert the shaft or needle 68. Additionally, or alternatively, audio and/or visual indications as to how to move the shaft or needle into the planned trajectory can be provided to the interventionist. Other examples of MR-guided interventions where the above described approach can be applied include, but are not limited to, MR-guided high-intensity focused ultrasound, MR-guided laser ablation, MR-guided radiofrequency ablation, MR-guided external beam radiotherapy, MR-guided brachytherapy, and so on. In the cases of MR guidance, the operator may have repositioned the patient intentionally during the intervention. Also, in those cases, the template scan used for the registration may have been acquired prior to the intervention during an examination used for planning the intervention. Further, images acquired from diagnostic scans advantageously maintain geometric alignment without exhibiting partial volume effects when there is patient motion between the diagnostic scans. During interventions, there is generally no time to perform post-processing steps, so it is beneficial to acquire the images directly in the desired geometry.

As used herein, a memory includes one or more of a non-transient computer readable medium; a magnetic disk or other magnetic storage medium; an optical disk or other optical storage medium; a random access memory (RAM), read-only memory (ROM), or other electronic memory device or chip or set of operatively interconnected chips; an Internet/Intranet server from which the stored instructions may be retrieved via the

Internet/Intranet or a local area network; or so forth. Further, as used herein, a processor includes one or more of a microprocessor, a microcontroller, a graphic processing unit (GPU), an application-specific integrated circuit (ASIC), an FPGA, and the like; a controller includes: (1) a processor and a memory, the processor executing computer executable instructions on the memory embodying the functionality of the controller; or (2) analog and/or digital hardware; a user input device includes one or more of a mouse, a keyboard, a touch screen display, one or more buttons, one or more switches, one or more toggles, voice recognition engines, and the like; a database includes one or more memories; and a display device includes one or more of a LCD display, an LED display, a plasma display, a projection display, a touch screen display, and the like.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A magnetic resonance (MR) system for maintaining geometric alignment of diagnostic scans during an examination of a patient, said system. comprising:
at least one processor programmed to:
in response to repositioning of the patient during the examination:
perform an updated survey scan of the patient;
select a scan completed during the examination as a template scan;
determine a transformation map between the template scan and the updated survey scan using a registration algorithm;
apply the transformation map to an initially defined scan geometry of a remaining diagnostic scan of the examination to produce an updated scan geometry;
generate a scan plan for the remaining diagnostic scan using the updated scan geometry; and,
perform the remaining diagnostic scan according to the scan plan.

2. The system according to claim 1, further including:
an MR scanner controlled to perform scans of the examination, including the updated survey scan, the template scan, and the remaining diagnostic scan.

3. The system according to claim 2, wherein the updated survey scan is a fast, three-dimensional survey scan.

4. (canceled)

5. The system according to claim 1, wherein the diagnostic scans are acquired during an MR-guided intervention.

6. The system according to claim 1, wherein the registration algorithm is chosen according to the MR contrast types of the template scan and the survey scan.

7. The system according to claim 1, wherein the at least one processor is further programmed to:
perform an initial survey scan, wherein the template scan is one of: (1) the initial survey scan; and (2) a diagnostic scan planned using the initial survey scan.

8. The system according to claim 1, wherein an MR contrast type of the updated survey scan is different than an MR contrast type of the template scan.

9. The system WO according to claim 1, wherein generating the scan plan includes:
displaying the updated survey scan with the updated scan geometry overlaid on the updated survey scan; and,
receiving acceptance of the updated scan geometry or modifications to the updated scan geometry.

10. The system according to claim 9, wherein the updated scan geometry is overlaid on the updated survey scan by displaying positions of corresponding slices.

11. The system according to claim 10, wherein the at least one processor is further programmed to:
automatically detect repositioning or strong motion of the patient.

12. A magnetic resonance (MR) method for maintaining geometric alignment of diagnostic scans during an examination of a patient, said method comprising:
in response to repositioning of the patient during the examination:
performing by at least one processor an updated survey scan of the patient;
selecting by the at least one processor a scan completed during the examination as a template scan;
determining by the at least one processor a transformation map between the template scan and the updated survey scan using a registration algorithm;
applying by the at least one processor the transformation map to
an initially defined scan geometry of a remaining diagnostic scan of the examination to produce an updated scan geometry;
generating by the at least one processor a scan plan for the remaining diagnostic scan using the updated scan geometry; and,
performing the remaining diagnostic scan according to the scan plan.

13. The method according to claim 12, further including:
performing scans of the examination, including the updated survey scan, the template scan, and the remaining diagnostic scan, using an MR scanner.

14. The method according to 12, wherein the updated survey scan is a fast, three-dimensional survey scan.

15. (canceled)

16. The method according to claim 12, wherein the diagnostic scans are acquired during an MR-guided intervention.

17. The method according to claim 12, furthering including:
performing an initial survey scan, wherein the template scan is one of: (1) the initial survey scan; and (2) a diagnostic scan planned using the initial survey scan;
wherein an MR contrast type of the updated survey scan is different than an MR contrast type of the template scan.

18. The method according to claim 12, wherein generating the scan plan includes:
displaying the updated survey scan with the updated scan geometry overlaid on the updated survey scan; and,
receiving acceptance of the updated scan geometry or modifications to the updated scan geometry.

19. The method according to claim 12, wherein further including:
automatically detecting repositioning or strong motion of the patient.

20. A magnetic resonance (MR) system for maintaining geometric alignment of diagnostic scans of a patient, said system comprising:
an MR scanner controlled to perform scans of the patient;
at least one processor programmed to:
perform an updated survey scan of the patient using the MR scanner
select a completed diagnostic scan of the patient as a template scan;
determine a transformation map between the template scan and the updated survey scan;
generate a scan plan for a subsequent diagnostic scan using the transformation map; and,
perform the subsequent diagnostic scan according to the scan plan.

21. The system according to claim 1, wherein the at least one processor is further programmed to perform an initial survey scan, wherein the template scan is a diagnostic scan planned using the initial survey scan.

22. The Method according to claim 12, furthering including performing an initial survey scan, wherein the template scan is a diagnostic scan planned using the initial survey scan.
